(19) United States
(12) Patent Application Publication
(10) Pub. No.: US 2012/0288485 A1
(43) Pub. Date: Nov. 15, 2012

(54) USE OF JASMONE FOR MODULATING MELATONIN PRODUCTION AND CALCIFICATION OF THE PINEAL GLAND

(75) Inventor: Brunde Broady, New York, NY (US)

(73) Assignee: Broady Health Sciences, LLC., Coppell, TX (US)

(21) Appl. No.: 13/467,606

(22) Filed: May 9, 2012

Related U.S. Application Data
(60) Provisional application No. 61/484,571, filed on May 10, 2011.

(51) Int. Cl. A61K 31/19; A61K 38/34; A61K 31/551; A61K 31/405; A61K 31/4045; A61K 36/16; A61K 31/565; A61K 31/7034; A61K 31/215; A61P 25/28; A61P 25/16; A61P 5/00; A61K 33/04; A61K 36/481; A61K 36/82; A61K 36/87; A61K 31/663; A61K 31/122; A61K 38/08

(52) U.S. Cl. 424/94.1; 514/21.6; 514/10.7; 514/221; 514/419; 514/415; 424/752; 514/171; 514/25; 514/557; 514/530; 514/9.7; 424/702; 424/757; 424/729; 424/766; 514/108; 514/690

(57) ABSTRACT

A formulation, composition or combination of substances comprising jasmonate for modulating melatonin production and/or calcification of a pineal gland and/or modulation and/or treatment of age-related neurodegeneration in a subject, particularly in a mammalian subject and more particularly in a human subject and use of jasmonate for modulating melatonin production and/or calcification of a pineal gland and/or modulation and/or treatment of age-related neurodegeneration is provided.

### TECHNICAL FIELD

A formulation, composition or combination of substances comprising jasmonate for modulating melatonin production and/or calcification of a pineal gland, in a subject, particularly in a mammalian subject and more particularly in a human subject and use of jasmonate for modulating melatonin production and/or calcification of a pineal gland is provided.

### BACKGROUND ART

### Melatonin

Melatonin is a hormone secreted by the pineal gland located in the center of the brain. Melatonin forms part of the system that regulates the sleep-wake cycle by chemically causing drowsiness. Besides its function as synchronizer of the biological clock, melatonin also exerts a powerful antioxidant activity. It is able to neutralize the highly toxic hydroxyl radical, the superoxide anion radical, and single oxygen radical. In addition, it increases levels of antioxidant enzymes including the stimulation of mRNA levels for superoxide dismustase and glutathione peroxidase, glutathione reductase and glucose-6-phosphate dehydrogenase. In certain sites, melatonin also reduces the pro-oxidant enzyme nitric oxide synthase. It also reduces lipid peroxidation and oxidative damage to DNA. (Reiter, R. 1998. Prog Neurobiol. 56: 359-389; Reiter, R. 1995. Exper. Gerontol. 30: 199-212; Venkataraman, et al. 2008. Int. J. Dev Neurosci 26: 585-591.)

Melatonin's antioxidant effect has been tested in a wide number of diseases related to oxidative damage. Melatonin has been shown to reduce amyloid protein toxicity of Alzheimer's disease (Pappolla, et al. 1997. Neurosci 17: 1683-90), to reduce delirium in elderly patients (Al-Aama et al. 2010. Int J of Ger Psychiatry ePub), to reduce oxidative damage in Parkinson's disease (Acuna-Castroviejo, et al. 1997. Life Sciences 60: 23-29). In addition, it has been shown to protect against glutamate excitotoxicity, and ischemia-reperfusion injury as well as hypertoxia, traumatic brain injury and neural toxins (Reiter, R. 1998. Prog Neurobiol. 56: 359-389).

Melatonin production is greatly reduced as a function of age and due to its antioxidant properties it has been suggested that reduced melatonin contributes to age related diseases. Supplemental melatonin has been shown to increase the life span of mice by 20% (Anisimov, et al. 2003. Exp Gerontol 38:449-61).

### Pineal Gland

An important factor in age related decline in melatonin is pineal calcification. This is likely the result of reduced Ca²⁺ ATPase levels which results in elevated intracellular calcium levels ultimately resulting in calcification (Chen, et al. 1993. Neuroscience Letter 157: 131-34.). Pineal gland function is thus of great importance in terms of aging and disease prevention. The pineal gland serves as a magnetoreceptor organ in the brain of humans and other mammals and its stimulation with an AC pulsed magnetic field has shown beneficial effects in the treatment of neurological and mental disorders which are associated with or related pathogenetically to impairment of pineal melatonin functions including multiple sclerosis, Parkinson's disease, juvenile Parkinsonism, progressive supranuclear palsy. Huntington's chorea, Shy-Drager syndrome, essential tremor, AIDS dementia complex, motor neuron disease, traumatic spinal cord injuries, ischemic stroke, diabetic neuropathy, dystonia, myoclonus, tardive dyskinesia, Tourette's syndrome, epilepsy, narcolepsy, Restless-legs syndrome, akathisia, chronic pain syndromes, migraine, Alzheimer's disease, depression (including seasonal affective disorder and premenstrual depression), autism, Attention Deficit hyperactivity disorder, schizophrenia, alcohol and substance abuse, obsessive-compulsive disorder, anxiety and panic disorder, posttraumatic stress disorder, trichotillomania, impulsive and aggressive behavior, chronic insomnia, sleep paralysis, and bulimia (see U.S. Pat. No. 5,885,976).

Studies have shown that melatonin release from the pinealocytes is calcium dependent (Zhao, et al. 1994. Mol Cell Endocrinol. 101: 189-96; Morton, et al. 1991. Proc Soc Exp Biol Med 197:378-83). Further studies have also shown that a deficiency in Ca²⁺ ATPase results in decreased pineal gland N-acetyltransferase (NAT) activity, resulting in a commensurate decline in pineal and serum melatonin levels (Reiter, et al. 1991. J Pineal Res. 11: 156-62).

### Jasmonates

Jasmonates are a family of plant stress hormones which are found in minute quantities in edible plants and characterized by cyclopentone rings. Various uses for jasmonates have been disclosed. Examples include enhancing plant growth (U.S. Pat. No. 5,436,226), repelling insects (U.S. Pat. No. 5,118,711), treating cancer (U.S. Pat. No. 6,469,061, US 20100003346) and treating skeletal muscle degeneration caused by malnutrition and disease (U.S. Pat. No. 6,465,021), pain relief (WO 2009019693), relieving psychological stress (US 2007/00420567), use as a component of a sleep supplement (JP2000355545), treating dry skin (US 20110085999, treating malodors on fabrics US 20110070181, improving cardiac muscle function (US 20110287116). Jasmonate has also been found to increase sarcoplasmic reticulum Ca²⁺ ATPase in cardiac and skeletal muscle sarcoplasmic reticulum (see, for example, Antipenko et al., 1997, J. Biol. Chem. 272:2852-60; Joumaa et al., 2002, J. Pharmacol. Exp. Ther. 300:638-46; Starling et al, 1995, Biochem. J. 308:343-6 and Starling et al., 1994, Biochemistry 15:3023-31).

### SUMMARY OF THE DISCLOSURE

Provided is a method for modulating melatonin production and/or calcification of a pineal gland in a subject in need thereof comprising administering an amount of jasmonate effective to modulate said melatonin production and/or said calcification. The method may also comprise administering at least one other substance that modulates melatonin production and/or calcification of the pineal gland. Such a subject may be a mammalian subject and particularly a human subject and in another embodiment, subject with age-related neurodegeneration.

In a related aspect, also provided are compositions and formulations comprising jasmonate and optionally a second substance used in modulating melatonin production and/or calcification of a pineal gland. In yet another related aspect, also provided is a combination of compounds comprising jasmonate and at least one other substance used in modulating melatonin production and/or calcification of the pineal gland. Such compositions, formulations or combinations may be used to modulate melatonin production and/or calcification of the pineal gland and particularly may be used to treat a subject with age-related neurodegeneration. Such a subject may be a mammalian subject and particularly a human subject, in another embodiment, subject with age-related neurodegeneration.

In yet another related aspect, provided are methods, compositions, formulations and combinations of substances for modulating or treating a subject with age related neurodegeneration comprising administering to a subject an amount of jasmonate and optionally at least one other substance that modulates or treats a subject with age-related neurodegeneration or compositions, formulations or combinations comprising said jasmonate and optionally said second substance effective to modulate or treat said age related neurodegeneration.

In a related aspect, also provided is the use of jasmonate and optionally one other substance, wherein said substance is a drug or natural substance used to modulate or treat a subject with age-related neurodegeneration and/or wherein said substance is used in modulating melatonin production and/or calcification of a pineal gland for modulating melatonin production and/or calcification of a pineal gland and/or for treating a subject with age-related neurodegeneration and/or formulating a medicament for melatonin production and/or calcification of a pineal gland and/or for treating a subject with age-related neurodegeneration.

### DETAILED DESCRIPTION OF SPECIFIC EMBODIMENTS

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

It must be noted that as used herein and in the appended claims, the terms “composition” and “formulation” are used interchangeably.

### Definitions

As defined herein, the term “modulate” means adjusting amount and/or rate of melatonin production and/or calcification of the pineal gland and/or age-related degeneration.

As defined herein, the terms “treat”, “treatment” and “treating” are to be understood accordingly as embracing prophylaxis and treatment or amelioration of symptoms of disease as well as treatment of the cause of the disease.

As defined herein, the term “age related neurodegeneration” refers to neurodegeneration resulting from disorders or diseases which aging is a major risk factor. Such disorders or diseases include but are not limited to dementia, Parkinson's disease, stroke.

As defined herein, “neurodegeneration” is the progressive loss of structure or function of neurons, including death of neurons.

### Jasmonates

The jasmonates used in the compositions and methods disclosed herein may have the formula I

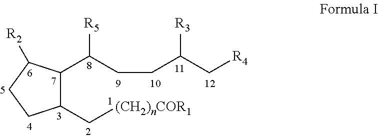

wherein:
n is 0, 1, or 2;
R₁ is OH, alkoxy, O-glucosyl, or imino,
R₂ is OH, O, alkoxy, or O-glucosyl,
R₃, R₄, and R₅ are H, OH, alkoxy or O-glucosyl,
and/or wherein R₁ and R₂, or R₁ and R₄ together form a lactone, and further wherein the bonds between C₃:C₇, C₄:C₅, and C₉:C₁₀ may be double or single bonds; or a derivative of said formula, wherein the derivative has at least one of the following:
   a lower acyl side chain at C₃ (free acid or ester or conjugate), a keto or hydroxy (free hydroxy or ester) moiety at the C₆ carbon, or an n-pentenyl or n-pentyl side chain at C₇.

In a particular embodiment, the jasmonate may be at least one member selected from the group consisting of methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-β-glucopyranoside, cucurbic acid-O-β-glucopyranoside 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids, and the carrier ligand conjugates and the sterioisomers thereof.

### Compositions

The compositions may comprise the jasmonate set forth above. Additionally, the compositions may further comprise least one other drug or natural substance used to modulate melatonin production and/or calcification of a pineal gland and/or modulating or treating age-related neurodegeneration. This drug or substance may include but is not limited to, melatonergic agents, noradrenergic, serotonergic re-uptake blockers, alpha-1-noradrenergic agonists, monamine oxidase inhibitors, neuropeptide Y agonists or antagonists; neurokinin-1 agonists; substance P; melanocyte stimulating hormone; beta-adrenergic blockers and benzodiazepines, such as atenolol; tricyclic antidepressants and alpha-2-adrenergic antagonists; melatonin precursors such as tryptophan, 5-hydroxytryptophan, serotonin and N-acetylserotonin; melatonin analogs (e.g., 6-chloromelatonin, 2,3-dihydromelatonin, 6-chloro-2,3-dihydromelatonin, N-acetyl-N-2-formyl-5-methoxy kynurenamine, N-acetyl-5-methoxy kynurenamine), melatonin agonists (e.g., melatonin, ramelteon and agomelatine) and melatonin antagonists.

The second substance may also include “cognitive drugs” which means any compound, composition, or drug useful for affecting cognitive function and include but are not limited monoamine oxidase B inhibitors such as selegiline; vasodilators such as nicerogoline and vinpocetine; phosphatidylserine; propentofyline; anticholinesterases (cholinesterase inhibitors) such as tacrine, galantamine, rivastigmine, vinpocetine, donepezil (ARJCEPT® (donepezil hydrochloride)), metrifonate, and physostigmine; lecithin; choline cholinomimetics such as milameline and xanomeline; ionotropic N-methyl-D-aspartate (NMDA) receptor antagonists such as memantine; anti-inflammatory drugs such as prednisolone, diclofenac, indomethacin, propentofyline, naproxen, rofecoxin, ibruprofen and suldinac; metal chelating agents such as cliquinol; Ginkgo biloba; bisphosophonates; selective oestrogen receptor modulators such as raloxifene and estrogen; a phytoestrogen; beta and gamma secretase inhibitors; cholesterol-lowering drugs such as statins; calcitonin; risedronate; alendronate; and combinations thereof. Cognitive drugs also include compositions known to affect cognitive function in animals such as those disclosed in published patent applications WO2009/045481, WO2010/014245, WO2007/070701, WO2007/041418, and WO2009/088433, and their equivalents in various countries and regions

The compositions may also include, but are not limited to, Astragalus or substances derived therefrom (e.g., astragaloside), gingerol, taurine, green tea or substances derived therefrom (e.g., epigallocatechin gallate), gingerol, taurine vitamin C, vitamin E, beta carotene and other carotenoids, selenium, lipoic acid, lycopine, lutein, zeaxanthin, coenzyme Q10, glutathione, N-acetyl cysteine, genistein, estradiol, and grape seed extract.

The compositions may comprise pharmaceutically acceptable salts of the active ingredients set forth above. The phrase “pharmaceutically acceptable salts” refers to derivatives of the above disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The compositions, in particular, pharmaceutical compositions can be formulated for administration by a variety of routes including but not limited to subcutaneous, topical, oral, intradermal, intramuscular, intraperitoneal, intravascular (e.g., intravenous), intra-arterial, intraventricular, and intracranial administration, intranasal, and epidural routes.

Such compositions are prepared in a manner well known in the pharmaceutical art and comprise as an active ingredient at least one of the compounds used in the methods as described herein above and a pharmaceutically acceptable carrier. The amount of the active ingredient(s) in the composition is from about 0.5 to 100% per weight. As used herein, the term “pharmaceutically acceptable carrier” refers to a carrier medium generally accepted in the art for the delivery of biologically active agents to animals, in particular, mammals, including, e.g., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents, and dispensing agents, depending on the nature of the mode of administration and dosage forms. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., as well known to those of ordinary skill in the art. The term “pharmaceutically acceptable” means approved by a regulatory agency of the Federal or a state government or listed in the U.S. Pharmacopeia or other generally recognized pharmacopeia for use in animals and, more particularly, in humans. Choosing suitable ingredients for the composition is a routine for those of ordinary skill in the art. It is evident that suitable carriers, solvents, gel forming ingredients, dispersion forming ingredients, antioxidants, colors, sweeteners, wetting compounds, release controlling components and other ingredients normally used in this field of technology may be also used.

The active ingredients may be formulated in the same pharmaceutical formulation. Furthermore, the compositions and formulations set forth herein may comprise one two or more jasmonates set forth above in separate compositions.

Alternatively, the active ingredients are formulated as separate pharmaceutical dosage forms. The combination of the pharmaceutical dosage forms may be packaged as a single medical product or kit for use in the method of the invention, optionally together with a package insert instructing to the correct use of the medical product.

### Administration and Uses

As noted above, one or more jasmonate(s) optionally in combination with other substances may be used to modulate melatonin production and/or pineal gland calcification. In a specific embodiment, the jasmonate optionally in combination with other substances may be used to treat age related neurodegeneration, neurodegeneration resulting from disorders or diseases in which aging is a major risk factor. Such disorders or diseases include but are not limited to dementia, Parkinson's disease, stroke. Dementia may result for example from Alzheimer's disease, vascular dementia and/or Dementia with Lewy bodies.

The active ingredients may be administered simultaneously, separately or sequentially. The administration routes of the active ingredients include, but are not limited to, subcutaneous, topical, oral, intradermal, intramuscular, intraperitoneal, intravascular (e.g., intravenous), intra-arterial, intraventricular, transdermal and intracranial administration, intranasal, and epidural routes.

The active ingredients furthermore may be administered as an immediate release formulation (a drug formulation that provides for release of the drug immediately after drug administration) controlled release formulation (a formulation in which release is not immediate) or a sustained release formulation (a drug formulation that provides for gradual release of a drug over an extended period of time, and that preferably, although not necessarily, results in substantially constant blood levels of a drug over an extended time period such as up to about 72 hours, about 66 hours, about 60 hours, about 54 hours, about 48 hours, about 42 hours, about 36 hours, about 30 hours, about 24 hours, about 18 hours, about 12 hours, about 10 hours, about 8 hours, about 4 hours, after drug administration). In a particular embodiment, the compositions may be administered prior to commencement of an activity where suppression of symptoms of an overactive bladder would be desirable.

The compositions used may, in a particular embodiment, be administered orally, preferably once per day. The suggested daily dose of jasmonate(s) is in general from about 0.01 to 50 mg, preferably from about 0.02 to 20 mg, more preferably from about 0.05 to 10 mg, and even more preferably, from about 0.05 mg-0.10 mg, depending on the age, body weight and condition of the patient. The effective amount of jasmonate(s) to be administered to a subject depends upon the condition to be treated, the route of administration, age, weight and the condition of the patient. Similar dosages of other substances may also be used.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

What is claimed is:

1. A method for modulating melatonin production and/or calcification of a pineal gland in a subject in need thereof comprising administering an amount of jasmonate effective to modulate said melatonin production and/or said calcification.

2. The method according to claim 1, wherein said calcification is modulated in said subject by modulating Ca²⁺ ATPase activity in the pineal gland of said subject.

3. The method according to claim 1, wherein said jasmonate has the structure (I) 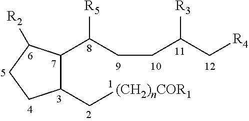
wherein:
n is0, 1, or 2;
R₁ is OH, alkoxy, O-glucosyl, or imino,
R₂ is OH, O, alkoxy, or O-glucosyl,
R₃, R₄, and R₅ are H, OH, alkoxy or O-glucosyl,
and/or wherein R₁ and R₂, or R₁ and R₄ together form a lactone, and further wherein the bonds between C₃:C₇, C₄:C₅, and C₉:C₁₀ may be double or single bonds; or a derivative of said formula, wherein the derivative has at least one of the following:
a lower acyl side chain at C₃ (free acid or ester or conjugate), a keto or hydroxy (free hydroxy or ester) moiety at the C₆ carbon, or an n-pentenyl or n-pentyl side chain at C₇.

4. The method according to claim 1, wherein said jasmonate is a compound selected from the group consisting of methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-b-glucopyranoside, cucurbic acid-O-b-glucopyranoside 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids, and the carrier ligand conjugates and the sterioisomers thereof.

5. The method according to claim 1, wherein said method further comprises administering at least one other substance that modulates said melatonin production and/or said calcification.

6. The method according to claim 5, wherein said other substance is selected from the group consisting of an anti-oxidant, monoamine oxidase inhibitors, substance P; melanocyte stimulating hormone; beta-adrenergic blockers; benzodiazepines; tricyclic antidepressants; alpha-2-adrenergic antagonists; melatonin precursors; melatonin analogs; melatonin antagonists, metal chelating agents, vasodilators and melatonin.

7. The method according to claim 6, wherein said other substance is selected from the group consisting of atenolol, tryptophan, 5-hydroxytryptophan, serotonin, N-acetylserotonin; ramelteon, agomelatine, vitamin C, vitamin E, beta carotene and other carotenoids, selenium, lipoic acid, lycopine, lutein, zeaxanthin, coenzyme Q10, glutathione, N-acetyl cysteine, melatonin, genistein, estradiol, Astragalus or substances derived therefrom (e.g., astragaloside), gingerol, taurine, green tea or substances derived therefrom (e.g., epigallocatechin gallate), gingerol, taurine, grape seed extract, selegiline; nicerogoline, vinpocetine; phosphatidylserine; cliquinol; Ginkgo biloba; bisphosophonates and calcitonin.

8. A method for treating a subject with age related neurodegeneration comprising administering to a subject in need thereof an amount of
(1) jasmonate and
(2) optionally at least one other substance used to treat age related neurodegeneration, effective to treat said age related neurodegeneration.

9. The composition to claim 8, wherein said age-related neurodegeneration is selected from the group consisting of dementia, Parkinson's disease and stroke.

10. A combination comprising (a) jasmonate and (b) a second substance used in modulating melatonin production and/or calcification of a pineal gland and/or age-related neurodegeneration selected from the group consisting of melatonergic agents, noradrenergic, serotonergic re-uptake blockers, alpha-1-noradrenergic agonists, monamine oxidase inhibitors, neuropeptide Y agonists or antagonists; neurokinin-1 agonists; substance P; melanocyte stimulating hormone; beta-adrenergic blockers and benzodiazepines, such as atenolol; tricyclic antidepressants and alpha-2-adrenergic antagonists; melatonin precursors such as tryptophan, 5-hydroxytryptophan, serotonin and N-acetylserotonin; melatonin analogs (e.g., 6-chloromelatonin, 2,3-dihydromelatonin, 6-chloro-2,3-dihydromelatonin, N-acetyl-N-2-formyl-5-methoxy kynurenamine, N-acetyl-5-methoxy kynurenamine), melatonin agonists (e.g., melatonin, ramelteon and agomelatine), melatonin antagonists and cognitive drugs selected from the group consisting of monoamine oxidase B inhibitors such as selegiline; vasodilators such as nicerogoline and vinpocetine; phosphatidylserine; propentofyline; anticholinesterases (cholinesterase inhibitors) such as tacrine, galantamine, rivastigmine, vinpocetine, donepezil (ARJCEPT® (donepezil hydrochloride)), metrifonate, and physostigmine; lecithin; choline cholinomimetics such as milameline and xanomeline; ionotropic N-methyl-D-aspartate (NMDA) receptor antagonists such as memantine; anti-inflammatory drugs such as prednisolone, diclofenac, indomethacin, propentofyline, naproxen, rofecoxin, ibruprofen and suldinac; metal chelating agents such as cliquinol; Ginkgo biloba; bisphosophonates; selective estrogen receptor modulators such as raloxifene and estrogen; a phytoestrogen; beta and gamma secretase inhibitors; cholesterol-lowering drugs such as statins; calcitonin; risedronate; alendronate; and combinations thereof.

11. The combination according to claim 10, wherein said combination is a composition.
